(19) United States
(12) Patent Application Publication
(10) Pub. No.: US 2010/0311176 A1
(43) Pub. Date: Dec. 9, 2010

(54) METHOD OF MASS ANALYSIS OF TARGET MOLECULES IN COMPLEX MIXTURES

(75) Inventors: Nicholas Williamson, Woodend (AU); Anthony Purcell, Victoria (AU)

Correspondence Address: KILYK & BOWERSOX, P.L.L.C., 3925 CHAIN BRIDGE ROAD, SUITE D401, FAIRFAX, VA, 22030, US

(73) Assignee: APPLIED BIOSYSTEMS, LLC, Foster City, CA (US)

(21) Appl. No.: 12/601,337

(22) PCT Filed: May 23, 2008
(86) PCT No.: PCT/IB08/02422

§ 371 Date: Jun. 24, 2010

(30) Foreign Application Priority Data
May 23, 2007 (AU) 2007902748

(51) Int. Cl. G01N 33/50; G01N 24/00

(52) U.S. Cl. 436/86; 436/173

(57) ABSTRACT

The present invention is a method for performing a mass spectrometric analysis of analytes in a complex mixture. In particular, samples containing unknown analytes are analyzed by MS/MS to identify portions of a molecule of interest that has been labeled with a selected isotope. Ionization and detection identify a characteristic isotope shift in real time based on a selective precursor ion scan that in turn identifies precursor masses that also contain the isotopic shift. From the precursor ion scan, precursor masses are identified for further mass spectrometric analyses. The method of the invention is preferably performed in real time such that the precursor ion scan simultaneously identifies target precursor ions and identifies precursor masses for further analyses.

### FIELD OF THE INVENTION

The present invention relates generally to methods for performing mass spectrometric analysis of target molecules samples and, in particular, to methods for performing mass spectroscopy analysis of samples to identify a precursor mass and identity using a precursor ion scan of a characteristic ion or ions.

### BACKGROUND OF THE INVENTION

Mass spectrometry is an analytical methodology used for qualitative and quantitative analysis of materials and mixtures of materials. In mass spectrometry, a sample of a material to be analyzed is introduced into the gas phase as ions. The particles are typically molecular in size. Once produced, the analyte particles are separated by the spectrometer based on their respective masses. The separated particles are then detected and a “mass spectrum” of the material is produced. The mass spectrum is analogous to a fingerprint of the sample material being analyzed. The mass spectrum provides information about the mass of the analytes and, in some cases, quantities of the various components that make up the material. In particular, mass spectrometry can be used to determine the molecular mass of molecules and molecular fragments within an analyte. Additionally, mass spectrometry can identify molecules of interest within the analyte based on the pattern of ionized fragments that are detected when the analyte is broken into fragments.

To determine the molecular mass of a compound, a number of different mass spectrometry instruments have been developed. The simplest forms include single quadrupole (Q) mass analyzers, time-of-flight (TOF) mass analyzers and ion trap (IT-MS) mass analyzers. For more complicated molecular structure analysis, however, instruments that can perform tandem mass spectrometry experiments (Tandem-MS or MS/MS) are often needed. Instruments capable of tandem mass spectrometry are typically more complex and often (though not exclusively, e.g., ion trap) combine multiple mass analyzers of the same or of different types, for instance triple quadrupole (QqQ), TOF-TOF MS, hybrid quadrupole linear ion trap (QqLIT) MS or hybrid quadrupole time of flight (Qq-TOF) MS. In tandem MS analysis, ionized particles are initially examined and then an ion of particular interest is selected. The selected ion is then isolated and fragmented using one of several different techniques, collisionally induced dissociation/collisionally activated dissociation (CID/CAD), electron captive dissociation (ECD), electron transfer dissociation (ETD), etc. The resulting fragments are then further characterized by mass analysis and the unique fragmentation pattern obtained is used to determine the structure of the corresponding analytes and/or molecules of interest.

For example, referring to FIGS. 1 and 7, a triple quadrupole (QqQ) mass spectrometer consists of three separate quadrupole sections connected together to act in concert. Each section acts independently of the other and different analytical methods can be selected by altering the function and performance of each section during a mass analysis. For example, the first quadrupole (Q1) can be used to select a particular precursor ion of a given mass. Then, the selected precursor or “parent” may be passed on to the second quadrupole (Q2) where the selected ion can be fragmented using collision induced dissociation, (CID), for example to yield product or “daughter” ions. These resulting product ions are then passed to the third quadrupole (Q3) which can then scan the fragments out to the detector for measurement. The resulting mass spectrum can be used to identify the product ions, which can be useful in identifying the structure of the selected precursor ion.

In the example described above, a single selected molecule is characterized. However, in many instances, the ion source may simultaneously produce multiple precursor ions requiring that each be analyzed individually. Most modern instruments can readily handle this situation and given sufficient time can systematically attempt to characterize one ion after another until all the available components are sampled. The disadvantage of this technique is that the time required by the machine to analyze each precursor, may be longer than the analyte is available in the case of very complex samples. For example, whenever samples are obtained by liquid chromatography (LC), the time is limited to the duration of the chromatographic peak in which the analyte(s) are contained during a LC-MS run.

An alternative to sampling all of the available precursors is to use some prior knowledge of the analyte of interest to selectively target molecules of interest. An example of this is a particular form of tandem mass spectrometry often referred to as “single ion monitoring” (SIM), “single reaction monitoring (SRM), or “multiple reaction monitoring” (MRM) (FIG. 1D). This technique can be used to monitor for the presence of a known compound in a mixture. SIM essentially uses the mass spectrometer to detect only molecules of a defined mass to charge ratio (m/z) allowing molecules of known mass to be identified with highest sensitivity. In complex mixtures it is desirable to use additional information about the targeted molecule to identify it from closely related molecules of similar or identical mass. Thus, SRM or MRM requires prior knowledge of the mass of the target molecule, in combination with knowledge of a characteristic fragment mass.

To specifically target both the known precursor and its respective product ions, using the example of the triple quadrupole mass spectrometer, the first quadrupole can be fixed to allow only ions with mass to charge ratio consistent with the target through to Q2. Thus, Q1 acts as a gate to specifically exclude all other precursor ions from the source. Q2 then fragments any ions that pass through Q1. The product ions are then passed to Q3 which is also fixed to target only a single product ion that is known to be diagnostic of the presence of the target molecule. The net effect of this is that the detector sees nothing until an ion enters the system that has a mass similar to the target molecule and can also produce a known product ion of the known target mass. This technique increases sensitivity and prevents wasting time on inspecting irrelevant product ions that are unrelated to the molecule of interest.

Neutral loss scanning (FIG. 1C) scans both analyzers in a synchronized manner, so that the mass difference of ions passing through MS1 and MS2 remains constant. The mass difference corresponds to a neutral fragment that is lost from a peptide ion in the collision cell. The neutral loss scan is therefore used to detect those peptides in a sample that contain a specific functional group. A common application of this method is the detection of peptides phosphorylated at serine or threonine residues via a loss of phosphoric acid.

Another variation on this experimental workflow is the “precursor ion scan” or “parent ion scan.” (FIG. 1B) In this example, prior knowledge of the molecule does not extend to knowledge of the precursor mass, but the precursor mass is known to contain a certain component that would result in the presence of a specific or diagnostic product ion. In this instance Q3 can be set to detect only the target product ion. Q1 is then used to scan across a mass range, allowing one ion at a time to sequentially enter Q2 where the ions are fragmented and then passed to Q3. Thus, in this example the detector “sees” nothing until Q1, scanning up through a mass range, passes a precursor that produces the target product ion. The net result is that the mass spectrometer is able to screen all of the incoming ions and selectively identify only those that contain the known functional group of interest. As in SIM/SRM/MRM, the advantage of this technique is a gain in sensitivity, with the advantage that time is not spent characterizing irrelevant ions. An overview of these various tandem mass spectroscopy experiments is present in FIGS. 1A-1D.

While precursor scanning and SIM/SRM/MRM are useful techniques, each is limited by the necessity to know in advance the precursor mass and/or the product ion mass for the class of molecules or the molecule of interest itself. These techniques have recently been reviewed; see, for example, Bruno Domon and Ruedi Aebersold, (2006), Mass Spectrometry and Protein Analysis, Science, 312, 212-217.

Despite the capabilities of these existing techniques in biochemical research, it is desirable to detect a distinct analyte from a mixture of other closely related analytes with a wide range of analyte concentrations. One example of a particularly difficult system to analyze is a peptide mixture, where it is often desirable to detect a distinct peptide from a mixture of closely related peptides.

The ability to detect fragments of unknown mass in complex mixtures is also important when molecules of interest are subjected to chemical or biochemical reactions in in vivo or in vitro assays that modify the molecule in unpredictable ways. For example, a polypeptide of interest may be reacted in a biochemical assay to determine how blood components, such as enzymes or other proteins, react with the polypeptide under physiological conditions. In such assays, a polypeptide molecule of interest, such as a protein, is injected into a research animal where modification of the polypeptide is caused by normal metabolic processes. Under such circumstances, the molecule of interest may be modified in unpredictable ways. The mass analysis of such a sample is difficult because the polypeptide of interest is processed in vivo and analysis of the metabolites of the original molecule would require separation of the polypeptide (or fragments thereof) of interest from the complex mixture of proteins, polypeptides, and other polypeptide fragments that would be present in any plasma or serum sample. Furthermore, under such circumstances, using chemical labels ordinarily used to identify exogenous polypeptides would not be available because the use of the label could alter the behavior of the molecule of interest in vivo and alter the modification(s) to the molecule of interest during the assay.

Separating a desired, distinct peptide from a mixture can readily be done using MRM's or precursor scans, but only if the mass of a fragment ion of the molecule of interest is well known in advance. Unfortunately, unexpected and unpredictable changes in mass occur due to many events, such as post-translational modification of the peptide, or the uncertainties generated by complex proteolysis of the parent protein or pro-polypeptide. This problem can be overcome to some extent by performing multiple precursor scans or MRMs, however, the peptides “discovered” will always be limited by the assumptions made for the respective experiments. i.e. requirement for a Q1 mass to be provided by the operator.

One approach to overcoming this problem has been by the use of partial isotopic labeling to enable the identification of target epitopes, see, for example, Hugo D. Meiring, Ernst C. Soethout, Martien C. M. Poelen, Dennis Mooibroek, Ronald Hoogerbrugge, Hans Timmermans, Claire J. Boog, Albert J. R. Heck, Ad P. J. M. de Jong and Cécile A. C. M. van Els; Table Isotope Tagging of Epitopes—A Highly Selective Strategy for the Identification of Major Histocompatibility Complex Class I-Associated Peptides Induced upon Viral Infection, Molecular & Cellular Proteomics, 5:902-913 2006. While they were able to identify several peptide epitopes, this method does not specifically screen the peptides in real time. Rather, a search engine is used to retrospectively interrogate the data to identify target peptides that were observed in the first run and then a second run was used to sequence the target peptide. This technique is cumbersome and not ideal due to the necessity to use multiple liquid chromatography (LC) runs for the same sample, thereby limiting the potential to identify low abundance peptides. For very complex samples there is also the complication of co-eluting isobaric peptides that would interfere with the identification process.

Another approach uses a precursor or “parent” ion scan for the immonium ion, see, for example Mathias Wilm, Gitte Neubauer, and Matthias Mann (1996) Parent Ion Scans of Unseparated Peptide Mixtures, Anal. Chem., 68:527-533 (1996). In this method, precursor ion scans are performed for immonium ions to identify the peptide precursor ion. The authors observed that the immonium ion can be a target for a precursor scan at improved detection limits and was proposed as a general tool for the detection of peptide precursors. The authors also observed that other low mass ions such as y ions could be used for the same purpose. They also demonstrated the utility of using low mass ions (m/z −79 and 204) as diagnostic markers in precursor scans for phosphorylated and glycosylated peptides. In each instance however, an assumption was made regarding the composition of the peptides to be targeted. For example, a precursor scan using the immonium ion of isoleucine/leucine would identify all isoleucine/leucine containing peptides. While useful, with regard to this application, the problem is that it cannot specifically target one isoleucine/leucine containing peptide in preference to any other.

It is therefore desirable to provide an analytical technique that can selectively screen out target analytes from a complex mixture in real time and does not require the same assumptions as a conventional MRM or precursor ion scan.

### SUMMARY OF THE INVENTION

The present invention provides a method for performing mass analysis of a molecule of interest. Using the method of the present invention, the analysis of a fragment of a molecule of interest does not depend on knowing the likely fragmentation pattern of the molecule of interest under any particular conditions. Instead, the identification of an isotope shift caused by an isotope introduced into the molecule of interest allows detection of a fragment of the molecule of interest allowing direct characterization of the precursor ion that generated the isotopically shifted fragment.

The method of the invention uses the isotopic labeling of a molecule of interest to distinguish and detect ions generated from the molecule during mass analysis. As noted above, when known molecules are subjected to fragmentation during mass analysis, the analysis of the resulting ions is typically performed by analyzing the resulting fragments that are known to result from ionization in the environment of mass analysis. However, where the fragmentation patterns are unknown, the analysis is more difficult or impossible.

The circumstances under which unknown fragments might exist in a sample mixture are also those where the analytical potential of mass analysis is greatest. Ideally, the mass analysis techniques and instrumentation would enable the researcher to obtain rapid and accurate analysis of specific ion fragments generated from a mixture of compounds contained in a sample mixture even where the fragmentation or ionization characteristics are not known in advance. However, this is not possible with current technology.

The present invention overcomes this drawback by introducing a selected isotope label into a molecule of interest, preferably a polypeptide, and then subjecting a sample mixture containing the isotope-labeled molecule to mass analysis where product ion fragments are characterized and analyzed. Because the invention is most valuable where the nature of the fragments are unknown, the invention can be used where a molecule of interest undergoes a chemical or biochemical reaction that results in unpredictable reactions yielding fragments, translations, phosphorylations, glycosylations, oxidation, reduction, sulfation, methylation, and any other form of post-translational modification or metabolite.

In the practice of the present invention, the methodology uses a precursor ion scan specific for a fragment containing the isotope incorporated into the molecule of interest. The precursor ion scan detects characteristic product ions or “daughter” ions that are unique to the fragment containing the selected isotope.

### BRIEF DESCRIPTION OF THE DRAWINGS

FIGS. 1A-1D. Schematic representation of various types of tandem mass spectroscopy experiments. FIG. 1A shows product ion scanning, FIG. 1B shows precursor ion scanning, FIG. 1C shows neutral loss scanning, Figure D shows multiple reaction monitoring.

FIG. 2. Illustrates an MS scan from a 70 minute LC/MS/MS experiment analyzing a preparation of eluted MHC peptides. The peaks represent a very complex set of peptides that are all eluting at the same time from the HPLC column.

FIGS. 3A and 3B. Illustrates the low mass region of a (product ion) MS/MS scan; (FIG. 3A) a normal unlabeled peptide; and (FIG. 3B) a ¹⁵N labeled peptide of the same sequence. The identified ¹⁵N immonium ions, for various amino acids are indicated with arrows.

FIGS. 4A-4E. Illustrates the potential of precursor ion scanning for ¹⁵N immonium ions to specifically screen out ¹⁵N labeled peptides from a mixture also containing unlabeled peptides. FIG. 4A shows a base peak chromatogram (BPC) for the mixture when analysed using a normal LC/MS/MS experiment—the peaks indicating the presence of peptides, both labeled and unlabeled. FIG. 4B-4E shows the result of using a precursor scan using ¹⁵N immonium ions as the diagnostic or target ion to screen the same mixture as in 4A. The peaks in each represent the intensity of the ¹⁵N immonium ion indicating the presence of a ¹⁵N labeled peptide precursor that must contain ¹⁵N labeled Valine (V), Glutamate (Q), Phenylalanine (F) or Lysine (K) respectively. In each instance the simplified profile demonstrates that labeled peptides were effectively “screened out” of the mixture and hence selected for further mass analysis.

FIG. 5. Identification of the ¹⁵N peptide EGVLYVGSK by precursor ion scanning for ¹⁵N immonium ions. From FIG. 4 above, the same peptide ¹⁵N EGVLYVGSK was identified in three of the four precursor scan experiments. FIGS. 5A,C, and E show the precursor ion intensity for ¹⁵N immonium ions for Valine (m/z 73), Glutamate (m/z) 103, and Lysine (m/z 131) respectively, while 5B, D, and F show resulting product ion spectra produced for the identified peptide ¹⁵N EGVLYVGSK. As expected (it does not contain phenylalanine), this peptide was not observed in the precursor scan using m/z 121 (phenylalanine). This demonstrates that multiple immonium ions may be used as diagnostic ions for the detection of the same peptide.

FIGS. 6A and 6B. Selective detection of the ¹⁵N peptide QGVAEAAGK from a BSA digest. A) FIG. 6 is the base peak chromatogram shows the complexity of the peptide mixture FIG. 6. B) the total ion chromatogram for a precursor scan using the immonium ion of glutamate and glutamine as a Q3 target mass. The peaks represent the presence of ¹⁵N labeled peptides. The BSA peptides in the sample were ignored. C) FIG. 6 the MS/MS spectrum of the ¹⁵N labeled peptide QGVAEAAGK

FIG. 7 is a schematic representation of possible alternative arrangements for performing methods according to the present invention. A) Using a triple quadrupole mass spectrometer. Analagous to a precursor scan Q1 selects one ion at a time and then passes to Q2 for fragmentation. Q3 then selects one or more product ions and scans them out to the detector. B) Using a triple quadrupole ion trap mass spectrometer. Analagous to a precursor scan Q1 selects one ion at a time and then passes to Q2 for fragmentation. Q3 then traps the product ions and scans them out to the detector. C) Using a triple quadrupole mass spectrometer. All ions or a range of ions are passed from Q1 to Q2 for fragmentation, Q3 then selects one or more product ions and scans them out to the detector. D) Using a triple quadrupole ion trap mass spectrometer. All ions or a range of ions are passed from Q1 to Q2 for fragmentation, Q3 then traps the product ions and scans them out to the detector. E) Using a hybrid QqTOF MS. Analagous to a precursor scan Q1 selects one ion at a time (or alternatively a range of ions) and then passes to Q2 for fragmentation. The product ions are then samples in the TOF section. When one or more precursor ions areas identified for further characterization the system swaps to regular MS/MS mode

### DETAILED DESCRIPTION OF THE INVENTION

### i. Definitions

Unless defined otherwise, all technical and scientific terms used herein generally have the same meaning as commonly understood by one of ordinary skill in the art to which this invention belongs. Generally, the nomenclature used herein and the laboratory procedures in molecular biology, organic chemistry described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references—(see generally, Sambrook et al. MOLECULAR CLONING: A LABORATORY MANUAL, 2^{nd} edition (1989) Cold Spring Harbor Laboratory Press, Cold Spring Harbor, N.Y.). The nomenclature used herein and the laboratory procedures in analytical chemistry, and organic synthetic described below are those known and employed in the art. Standard techniques, or modifications thereof, are used for chemical syntheses and chemical analyzes.

As used herein “assay” refers to a process whereby an isotopically labeled molecule of interest is reacted in a test or analytical method designed to measure a structural change in the molecule of interest or derivative thereof, particularly where the molecule of interest undergoes modifications under in vitro or in vivo conditions where the outcome is uncertain. For example, in an “assay” of the invention, a molecule of interest such as a protein or polypeptide is isotopically labeled and tested in an assay where the isotope labeled polypeptide is metabolized, fragmented, cleaved, truncated, modified, glycosylated, phosphorylated, or otherwise modified. Typically, the assay yields a sample wherein the modified, isotopically labeled molecule is present with a mixture of other analytes inherent in the assay, including other proteins and polypeptide fragments from which separation or identification of the portion of the molecule of interest would be difficult or impossible by traditional mass analysis. An advantage of the present invention is the ability to detect fragments or modifications to the labeled molecule of interest resulting from the assay and that cannot be predicted in advance.

As used herein, “molecule” refers to a molecule of interest. Non-limiting examples of molecules include, but are not limited to, amino acid, protein, a polypeptide comprising one or more amino acids in linear or branched configuration, a polypeptide fragment, a peptide analog partial or complete, a protein in any isoform or fragment, an antibody of any type, a, nucleic acid (both DNA or any RNA), a carbohydrate, lipid, steroid and other biomolecule. Molecule could also refer to any synthetic molecule such as polymers or other molecule where an isotope could be used to replace a constituent atom of the compound and the labeled compound subjected to mass analysis. The source of the molecule, or the sample comprising the molecule, is not a limitation as it can come from any source and can be natural or synthetic. Non-limiting examples of sources for the molecule, or the sample comprising the molecule, include cells or tissues, or cultures (or subcultures) thereof. Non-limiting examples of molecule sources include, but are not limited to, crude or processed cell lysates, body fluids, vaccines, tissue extracts, cell extracts or fractions (or portions) from a separations process such as a chromatographic separation, a 1D electrophoretic separation, a 2D electrophoretic separation or a capillary electrophoretic separation. Body fluids include, but are not limited to, blood, urine, feces, spinal fluid, cerebral fluid, amniotic fluid, lymph fluid or a fluid from a glandular secretion. The cell lysates are processed or is treated, in addition to the treatments needed to lyse the cell, to thereby perform additional processing of the collected material. For example, the sample can be a cell lysate comprising one or more biomolecules that are peptides formed by treatment of the cell lysate with a proteolytic enzyme to thereby digest precursor peptides and/or proteins. Similarly, the peptide can be created through known synthetic techniques where the ¹⁵N isotope is introduced during synthesis.

As used herein, “labeled,” “isotope” or “isotopically labeled” means that a constituent atom of a molecule of interest has been replaced with one or more atom isotopes (e.g. isotopes such as ²H, ³H, ¹³C, ¹⁴C, ¹⁵N, ¹⁸O, ³²P, ³³P, ³⁵S, ³⁷Cl, ¹²⁵I or ⁸¹Br). The isotope may be a heavy isotope or a light isotope. Because isotopic labeling is not 100% effective, a composition comprising the isotope-labeled molecule of interest may still contain impurities of the compound that are of lesser states of enrichment and these will have a lower mass. Likewise, because of over-enrichment (undesired enrichment) and because of natural or unlabeled isotopic abundance, there can be impurities of greater mass.

As used herein, an “isotopic shift” is a difference in the molecular mass of two molecules or ions (such as two peptides, or peptide ions) that can be calculated from the molecular formulas and isotopic contents of the two molecules or ions. An isotopic shift is present between two molecules or ions of the same formula when a known number of atoms of one or more type in one molecule or ion are replaced by lighter or heavier isotopes of those atoms in the other molecule or ion. For example, replacement of a ¹²C atom in a molecule with a ¹³C atom (or vice versa) provides an isotopic shift of about 1 atomic mass unit (amu), replacement of a ¹⁴N atom with a ¹⁵N atom (or vice versa) provides an isotopic shift of about 1 amu, and replacement of a ¹H atom with a ²H (or vice versa) provides an isotopic shift of about 1 amu. The differences in mass that comprise an isotopic shift between the masses of particular atoms in two different molecules or ions are summed over all of the atoms in the two molecules or ions to provide an isotopic shift between the two molecules or ions.

As used herein, the “unlabeled” is the molecule of interest lacking and isotope labeling or enrichment and which is expected to contain atoms of the most abundant or natural isotope for each element and/or isotopes lower than a threshold amount representing the natural background concentration. This may be referred to as the natural molecule.

As used herein, “intensity” refers to the height of, or area under a peak representing the concentration on abundance of an analyte. For example, the peak can be output data from a measurement occurring in a mass spectrometer (e.g., as a mass to charge ratio (m/z)). In accordance with some embodiments of the present invention, intensity information can be presented as a maximum height of the peak or a maximum area under the summary peak representing a mass-to-charge ratio.

The creation of the isotope labeled equivalent or analog of the molecule of interest replaces a constituent atom at the molecule with a selected isotope. This approach yields an isotope-labeled molecule that is substantially identical to the molecule of interest but differs in mass by the sum of the differences in mass between the isotope between the isotope and the natural element. This technique has the further advantage compared, for example, to using a bulky mass tag, of causing the isotope-labeled molecule to behave the same as the molecule of interest when measured or manipulated after labeling. This further advantage is particularly beneficial where a biomolecule is studied as the molecule of interest. An isotope labeled “version” as the molecule of interest, such as a protein is synthesized in the presence of a selected isotope to substantially replace the natural or native constituent atom with the isotope. After synthesis, the protein has the sum structure but contains, for example ¹⁵N, replacing the non-isotopic ¹⁴N that would normally exist. Accordingly, for each N atom in the protein the mass is increased by one. For a characterized protein ion fragment ion containing a single ¹⁵N atom, the mass is increased by one (See Table 1 below).

Thus, in a preferred embodiment, the molecule of interest is a polypeptide with an isotope integrated into the molecule during expression or synthesis such that the precursor ion scan detects ionized peptide fragments having a unique mass signature. Once such a characteristic ion is detected, the “parent” or precursor mass is identified as having originated with the molecule of interest prior to being introduced into a sample mixture. In this process, detecting the characteristic precursor ion by the unique mass signature, and using this unique signature to identify the precursor mass in real time, the methodology of the invention reduces the number of precursor mass peaks that must be examined to identify a precursor mass of the molecule of interest. Because the characteristic product ion or “daughter ion” permits ready identification of the precursor mass, the mass spectrometric machinery can be tasked to perform a mass analysis of only these precursor masses so identified by the precursor ion scan without wasting valuable machine time or limited sample on analytes that are unrelated to the molecule of interest. Furthermore, by measuring the intensity and abundance of the isotope, background isotope levels or coincidental overlap of other analytes in the precursor ion scan can be eliminated and a real-time scan of only the critical precursor mass data can be performed.

Accordingly, the present invention provides a mass spectrometric method for analysis of a sample containing a molecule of interest, advantageously when the sample comprises a plurality of molecules, at least one of which is isotopically labeled, the method comprising the steps of:
(a) labeling a molecule of interest with an isotope;
(b) reacting the labeled molecule in an assay wherein the molecule undergoes a chemical or biochemical reaction expected to modify the molecule;
(c) selecting a molecule from the sample by subjecting it to ionization and fragmentation to produce fragments thereof;
(d) performing a precursor ion scan specific for ions containing the isotope determining the presence of at least one isotopically labeled fragment of the molecule of interest in real time and optionally;
   i) if at least one isotopically labeled fragment is present, subjecting the isotope-labeled molecule to further mass spectrometric analysis; and, optionally, performing steps (c) and (d) with a further molecule from the mixture;
   ii) if a labeled fragment ion is not present, performing steps (c) and (d) with a further molecule from the mixture.

The present invention also provides a method of analysis of any modification of an amino acid, peptide or protein, the method comprising:
(a) introducing an isotope label to the amino acid, peptide or protein molecule, subjecting the labeled amino acid, peptide or protein from the sample and subjecting it to ionization and fragmentation to produce fragments thereof;
(b) reacting sample comprising a plurality of amino acids, peptides or proteins, and the isotopically labeled amino acid, peptide or protein;
(c) determining the presence of at least one isotopically labeled fragment of the labeled amino acid, peptide or protein in a precursor ion scan;
(d) determining the presence of at least one isotopically labeled fragment and performing further mass analysis; and
   i) wherein the step of determining if at least one isotopically labeled fragment is present, subjecting the molecule to further mass spectrometric analysis; and, optionally, performing steps (c) and (d) with a further molecule from the mixture; and/or
   ii) if a labeled fragment ion is not present, performing steps (c) and (d) with a further fragment from the mixture; and
(e) determining a modification of the amino acid, peptide or protein from step (a).

The modified amino acid, peptide or protein may be purified prior to conducting the analysis. The modification can be the result of metabolism and/or one or more post-translational modification, phosphorylations, glycosylations, cleavages, truncation, fragmentations or any other structural change causing a change in mass to any compound.

The present invention also includes machine-readable medium having stored thereon a plurality of executable instructions to perform a mass spectrometric method of analysis of a sample, the sample comprising a plurality of molecules, at least one of which is isotopically labeled, the method comprising the steps of:
(a) designating a selected isotope used as a label, wherein the designation includes identifying the characteristic isotopic shift of preselected isotope;
(b) selecting a molecule from the sample by subjecting it to ionization and fragmentation to produce fragments thereof;
(c) determining the presence of at least one isotopically labeled fragment by detecting the characteristic isotope shift of the selected isotope;
   i) if at least one isotopically labeled fragment is present, subjecting the molecule to further mass spectrometric analysis; and, optionally, performing steps (b) and (c) with a further molecule from the mixture;
   ii) if a labeled fragment ion is not present, performing steps (b) and (c) with a further molecule from the mixture.

The present invention also provides a tandem mass spectrometric method of identifying a target precursor molecule in sample, the sample comprising a plurality of molecules at least one of which is isotopically labeled, the method comprising the steps of:
(a) selecting a molecule from the sample and subjecting it to ionization and fragmentation to produce fragments thereof;
(b) determining the presence of at least one isotopically labeled fragment;
   i) if at least one isotopically labeled fragment is present, using the mass-to-charge ratio of the selected molecule as a target precursor in a further tandem mass spectrometric analysis; and, optionally, performing steps (a) and (b) with a further molecule from the mixture;
   ii) if a labeled fragment ion is not present, performing steps (a) and (b) with a further molecule from the mixture.

The present invention also provides a method of analyzing or comparing the intensity of the signal from a characteristic product ion derived from the isotopic label and comparing the intensity to a background level of the isotope to prevent confusion at the natural isotopic abundance of an element with detection of a labeled molecule created as part of the method of the invention. This approach reduces the chance of the erroneous selection of a precursor mass for further mass analysis based on a trace or background intensity of the isotope. This aspect of the method may include comparing the intensity of either the regular (i.e. unlabeled) fragment and that of the corresponding isotopically labeled fragment or both against a threshold level. The threshold level can be a value arbitrarily selected by the mass analysis researcher or can be based on any signal to noise that excludes trace levels of the isotope. Preferably, the threshold level is a ratio of the intensity of target ion against a background level of the isotope such that measurements of the known isotopic abundance of the isotope is excluded from further mass analysis.

In one example, the monitoring may be performed using a software algorithm that monitors the ratio of the regular immonium ion peak intensity and the corresponding intensity of the ¹⁵N (or other isotope) immonium ion. A greater than 1% change in the ratio of isotopic abundance due to the presence of a ¹⁵N immonium ion maybe sufficient to indicate the presence of a ¹⁵N labeled fragment.

Additionally, the intensity or abundance of a target ion that lacks the isotope can be measured to facilitate the identification of the target precursor mass. In the example of ¹⁵N immonium ion the unlabeled immonium ion peak intensity of an amino acid, peptide or protein and the corresponding intensity of the ¹⁵N labeled immonium ion so as to allow real time selection of target precursor ions.

The present invention also includes using pattern recognition software to detect changes in the unlabeled immonium ion peak intensity of an amino acid, peptide or protein and the intensity of the ¹⁵N labeled immonium ion of an amino acid, peptide or protein so as to allow real time selection of target precursor ions.

The present invention also includes using any mathematical means that can detect a change in the isotope abundance (particularly in the low mass region) so as to allow real time selection of target precursor ions. The present invention also provides a method of using any mathematical means that can discern a change in the unlabeled product ion intensity and the corresponding intensity of the isotopically labeled product ions so as to allow real time selection of target precursor ions.

The present invention also includes analyzing or comparing the unlabeled product ion intensity and the corresponding intensity of the isotopically labeled product ions as part of the precursor ion scan to allow real time selection of target precursor ions and the subsequent use of a target precursor ion to allow real time identification of a precursor mass.

In one example the monitoring may be performed using a software algorithm that monitors the ratio of the regular immonium ion peak intensity and the corresponding intensity of the ¹⁵N (or other isotope) immonium ion. A greater than 1% change in the ratio of isotopic abundance due to the presence of a ¹⁵N immonium ion maybe sufficient to indicate the presence of a ¹⁵N labeled fragment.

The step of performing a precursor ion scan may simultaneously detect the presence of one or more than one isotopically labeled precursor from the molecule of interest. Thus in the practice of the present invention, simultaneously monitoring of more than one of the isotopically labeled product ions may provide a much greater chance of identifying all of the isotopically labeled precursor molecules in the sample. An additional benefit of this would be that by using multiple target ions one reduces the possibility of false positives due to chemical noise, difference in the molecule, for example peptide, concentrations in the sample, or other contaminants in the sample.

As described herein, the isotope labeled molecule of interest may be exposed or reacted in an in vivo or in vitro assay. The method of the invention is particularly valuable where the assay is designed to mimic a biological system that causes modification to the labeled molecule in the same manner as the molecule of interest, including but not limited to enzymatic cleavage or fragmentation where the labeled molecule and the molecule of interest are polypeptides. In most such assays, the modifications that are created in the molecule of interest are not predictable and not known in advance, as when a sample mixture from the assay containing the labeled molecule is subjected to mass spectrometric analysis. The fragment or modified species of the molecule of interest will not be identifiable from a characteristic signature in a precursor ion scan absent the identification of the isotope label. Rather, the precursor ion scan identifies characteristic ions corresponding to the isotopic shift caused by the label introduced into the molecule of interest. As noted above, if the intensity is determined to fall above or below a detection threshold that may reflect the abundance of the introduced isotope such that the intensity falls above the detection threshold. This indicates that the precursor ion scan has detected an isotopic shift at an intensity that distinguishes it from the natural abundance of the isotope. If so, the detection of the characteristic product ions in the precursor ion scan identifies a precursor mass that is necessarily derived from the labeled molecule of interest. At this point, an enhanced resolution scan of the identified precursor mass is performed together with or independent from a regular product ion scan of the identified precursor mass, for example, to sequence a polypeptide identified as a precursor mass derived from the molecule of interest. The enhances resolution scan(s) regular product ion scans can be formed independently and in any order.

In another embodiment, the molecule of interest is labeled as described herein with a known isotope. Following the assay, a precursor scan is performed to monitor the intensity of two or more characteristic product ions. After determining that each intensity is above the threshold level, a single precursor mass derived from the labeled molecule is identified and analyzed by conventional methods.

Additionally, the presence of multiple product ions identified in the precursor ion scan may preferentially select an individual precursor mass for analysis. Thus, if multiple target or product ions identify the same precursor mass as a result of the isotopic shift, then this specific precursor mass is identified characterized preferentially to other precursor masses that may be identified from other precursor ion scans. Preferentially identified precursor masses are then identified through enhanced resolution scans of the identified precursor mass.

In another embodiment, in addition to monitoring the specificity and intensity of unique characteristic product ions, the characteristic product ions can be monitored together with the regular unlabeled ions that would be expected in any precursor scan. For example, if ¹⁵N were used as the isotope for labeling the molecule of interest, the method of the invention includes monitoring the intensity of ions containing both ¹⁵N and corresponding regular ¹⁴N atoms. The ratio of the intensity is used to determine if the regular precursor mass, identified by the precursor ion scan, was ¹⁵N labeled during the first step of the process. If the ¹⁵N characteristic ion signature is identified, then the identified corresponding precursor mass is subject to an enhanced resolution scan at the identified precursor mass together with or independent from a regular product ion scan.

As will be apparent to those skilled in the art, the use of any particular precursor ion scan, the comparison of a background or threshold level of the isotope to avoid false positives as described herein, the use of a plurality of precursor ion scans and various measurements thereof and/or the preferential selection of precursor masses from a plurality of precursor ion scans are all independent steps that can be selectively used either alone or in combination with the other techniques described herein to practice the fundamental method of the invention.

Although measurement of an ¹⁵N immonium ion is described here, it should be noted in addition to the immonium ions, other target ions can be used, for example, any of the list of a1, b1, c1, x1, y1, and z1 ions could also readily be used as characteristic product ions in a precursor ion scan pursuant to the methodology of the invention.

Similarly, although the ¹⁵N isotope is used herein, the molecule of interest may be isotopically labeled with any isotope including one selected from ²H, ³H, ¹³C, ¹⁴C, ¹⁵N, ¹⁸O, ³²P, ³³P, ³⁵S, ³⁷Cl, ¹²⁵I or ⁸¹Br. As noted herein, ¹⁵N and ¹⁴C are preferred for polypeptide analysis (see Table 1 and 2) and ¹⁵N is most commonly used based on cost.

The isotopically labeled fragment may be a low mass fragment. The low mass fragment may have a mass-to-charge ratio of less than about 250.

The molecule of interest may be an amino acid, protein, a polypeptide comprising one or more amino acids in linear or branched configuration, a polypeptide fragment, a peptide analog partial or complete, protein in any isoform or fragment, an antibody of any type, a nucleic acid (both DNA or any RNA), carbohydrate, lipid, steroid and other biomolecule.

As noted above, the characteristic product ion or “daughter” ion can be selected from several different ions e.g., a1, b1, c1, x1, y1, and/or z1 or the immonium ion or immonium related ions and may include essentially any isotope that is readily detected by mass analysis. For the identification of compounds of interest comprised of proteins, peptides, polypeptides, or other forms of amino acids, the isotopes of nitrogen and carbon are preferred due to their abundance in the atomic structure of these molecules. The ¹⁵N isotope is specifically preferred in polypeptide due to its ready availability, cost, and ease of incorporation into the peptide molecule during synthesis through well known and readily available expression techniques.

The isotopically labeled fragment maybe selected from any one or more of the following isotopically labeled amino acids:

TABLE 1
List of immonium ions and the corresponding ¹⁵N labeled
immonium ions, bold face indicated strong signal

| Residue 3-letter | 1-letter | Immonium | Related | ¹⁵N Immonium | ¹⁵N Related |
|---|---|---|---|---|---|
| code | code | ion | ions | ion | ions |
| Alanine, Ala | A | 44 | | 45 | |
| Arginine, Arg | R | 129 | 59, 70, 73, 87, | 133 | 103, 89, 74, |
| | | | 100, 112 | | 114, 71, 60 |
| Asparagine, Asn | N | 87 | 70 | 89 | 71 |
| Aspartic acid, Asp | D | 88 | 70 | 89 | 71 |
| Cysteine, Cys | C | 76 | | 77 | |
| Glutamic acid, Glu | E | 102 | | 103 | |
| Glutamine, Gln | Q | 101 | 56, 84, 129 | 103 | 85, 131 |
| Glycine, Gly | G | 30 | | 30 | |
| Histidine, His | H | 110 | 82, 121, 123, | 113 | 169, 141, 84, |
| | | | 138, 166 | | 125, 123 |
| Isoleucine, Ile | I | 86 | 44, 72 | 87 | 73, 45 |
| Phenylalanine, Phe | F | 120 | 91 | 121 | |
| Proline, Pro | P | 70 | | 71 | |
| Leucine, Leu | L | 86 | 44, 72 | 87 | 45, 73 |
| Lysine, Lys | K | 101 | 70, 84, 112, | 103 | 131, 114, 85 |
| | | | 129 | | |
| Methionine, Met | M | 104 | 61, 70 | 105 | |
| Serine, Ser | S | 60 | | 61 | |
| Threonine, Thr | T | 74 | | 75 | |
| Tryptophan, Trp | W | 159 | 77, 117, 130, | 161 | 172, 173, |
| | | | 132, 170, 171 | | 118, 131, 133 |
| Tyrosine, Tyr | Y | 136 | 91, 107 | 137 | |
| Valine, Val | V | 72 | 41, 55, 69 | 73 | 56, 70 |

TABLE 2
List of immonium ions and the corresponding ¹³C labeled
immonium ions

| Residue | | | |
|---|---|---|---|
| 3-letter code | 1-letter code | Immonium ion | ¹³C Immonium ion |
| Alanine, Ala | A | 44 | 46 |
| Arginine, Arg | R | 129 | 134 |
| Asparagine, Asn | N | 87 | 90 |
| Aspartic acid, Asp | D | 88 | 91 |
| Cysteine, Cys | C | 76 | 78 |
| Glutamic acid, Glu | E | 102 | 106 |
| Glutamine, Gln | Q | 101 (84) | 105 (88) |
| Glycine, Gly | G | 30 | 30 |
| Histidine, His | H | 110 | 115 |
| Isoleucine, Ile | I | 86 | 91 |
| Phenylalanine, Phe | F | 120 | 128 |
| Proline, Pro | P | 70 | 74 |
| Leucine, Leu | L | 86 | 91 |
| Lysine, Lys | K | 101 | 106 |
| Methionine, Met | M | 104 | 108 |
| Serine, Ser | S | 60 | 62 |
| Threonine, Thr | T | 74 | 77 |
| Tryptophan, Trp | W | 159 | 169 |
| Tyrosine, Tyr | Y | 136 | 144 |
| Valine, Val | V | 72 | 76 |

Optionally, the sample may be fractionated and/or purified to select analyses prior to performing the analysis. The sample may be purified by liquid chromatography. In particular, the sample may be purified by high pressure liquid chromatography.

Dissociation of an analyte may comprise any one or more of a method selected from (i) collisions with an inert gas (collision-induced dissociation (CID) or collisionally-activated dissociation (CAD)); (ii) collisions with a surface (surface-induced dissociation (SID)); (iii) interaction with photons resulting in photodissociation, optionally using a laser; (iv) thermal/black body infrared radiative dissociation (BIRD); and (v) interaction with an electron beam, resulting in electron-induced dissociation for singly charged cations (EID), electron-capture dissociation (ECD) and electron-transfer dissociation (ETD) for multiply charged cations, or combinations thereof.

In one embodiment, the method may be used for analysis of the modification of the molecule of interest in an assay that mimics modifications that occur to the molecule in a biological system. In particular, the method may be used for analysis of the metabolism or post-translational modification status of an amino acid, peptide or protein.

The method may be used to identify a peptide epitope. The method may also be used to identify cell derived biomarker.

The mass spectrometric analysis may be performed on a high resolution mass spectrometer or by tandem mass spectrometry.

The tandem mass spectrometer may be equipped with electrospray ionization (ESI) or matrix assisted laser desorption ionization (MALDI) interfaces to transfer the target precursor ion into the gas-phase.

The mass spectrometric analysis may be performed using a mass spectrometer selected from the group comprising a triple quadrupole, 3D or linear ion trap, TOF-TOF MS, QqLIT MS, Qq-TOF MS, QqtrapTOF, LIT-orbitrap or LIT-FT-ICR.

The tandem mass spectrometer may be a tandem-in-space mass spectrometer, a tandem-in-time mass spectrometer, or a combination thereof.

The tandem-in-space mass spectrometer may be a sector mass spectrometer, a time of flight mass spectrometer, a triple quadrupole mass spectrometer, or a hybrid mass spectrometer combining time of flight and quadrupole instruments.

The sector mass spectrometer may be a double focusing sector mass spectrometer or a hybrid mass spectrometer combining sector and quadrupole instruments.

The tandem-in-time mass spectrometer may be a two-dimensional quadrupole ion trap mass spectrometer, a three-dimensional quadrupole ion trap mass spectrometer or a Fourier-transform ion cyclotron resonance (FT-ICR) mass spectrometer.

The tandem-in-space mass spectrometer may be a sector mass spectrometer, a time of flight mass spectrometer, a triple quadrupole mass spectrometer, or a hybrid mass spectrometer combining time of flight and quadrupole instruments.

The sector mass spectrometer may be a double focusing sector mass spectrometer or a hybrid mass spectrometer combining sector and quadrupole instruments.

The tandem-in-time mass spectrometer may be a two-dimensional quadrupole ion trap mass spectrometer, a three-dimensional quadrupole ion trap mass spectrometer or a Fourier-transform ion cyclotron resonance (FT-ICR) mass spectrometer.

### ii. Isotopically Labeled Molecules

Stable isotopes may be incorporated into molecules either biologically (in vivo) or chemically (in vitro) through synthesis to yield an isotope labeled molecule of interest.

Biological isotopic labeling of peptides can be achieved by growing an expression host in a growth media enriched with the desired isotopes. A number of well known organisms are capable of growing on a defined minimal medium, containing a source of the desired isotope, e.g. ¹⁵N ammonium chloride or ¹⁵N ammonium sulfate and incorporating the selected isotope into the polypeptide. Suitable organisms include organisms such as Escherichia coli, which is the most frequently used bacteria, and Pichia pastoris, which is the most frequently used yeast. Insect and mammalian cells that are especially selected for expression of the molecule of interest and can also be grown in labeled media. The expressed labeled protein is then purified from the organism for later use. Examples of useful techniques labeling proteins are described in Methods in Molecular Biology: Protein NMR Techniques, Edited by Kristina Downing, Humana Press Inc., 2nd Rev. Edition (August 2004).

Chemical isotopic enabling of any compound can also be achieved by any number of known techniques whereby an isotope is incorporated into a compound during chemical synthesis or is later substituted into a compound to replace a constituent atom of the compound. As in the case of a peptide or other biological molecule, an isotopic shift is present between two molecules when a number of known constituent atoms of one or more types in a molecule are replaced by light or heavy isotopes of those atoms in a molecule. The resulting isotopic shift is comprised of the differences in mass attributable to the sum of the differences of the masses of the constituent atoms and the replacement isotopes.

### iii. Identifying a Subset of Target Peptides from a Sample Containing Mixture of Other Peptides by Identifying a Unique Precursor Mass from a Precursor Ion Scan of Product Ions

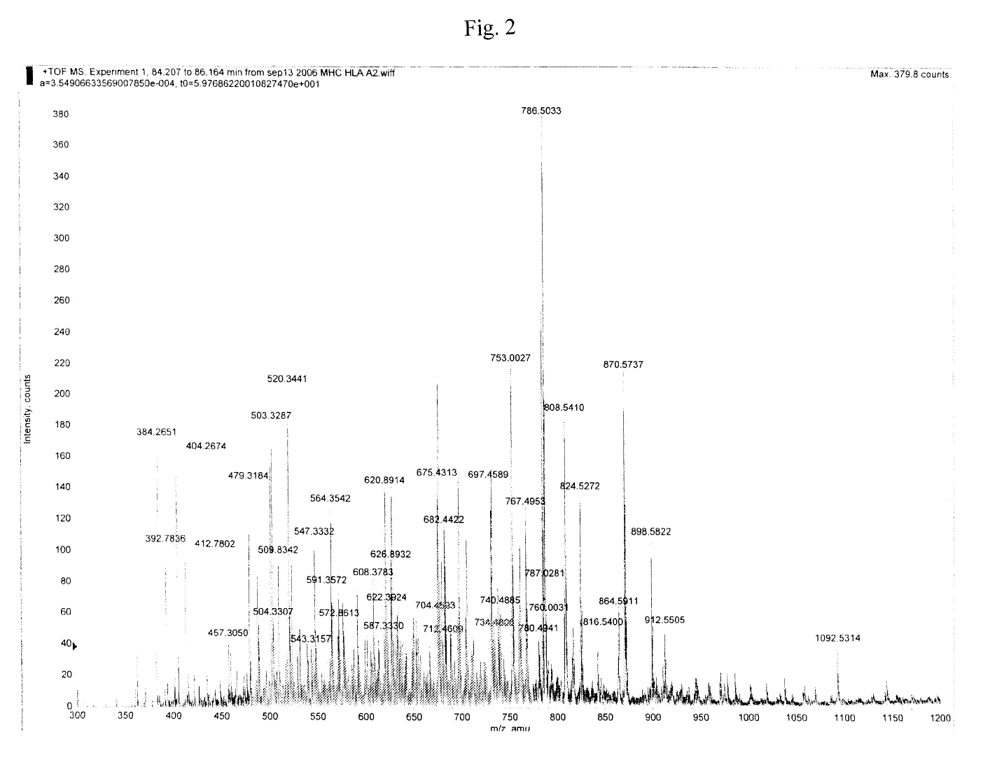

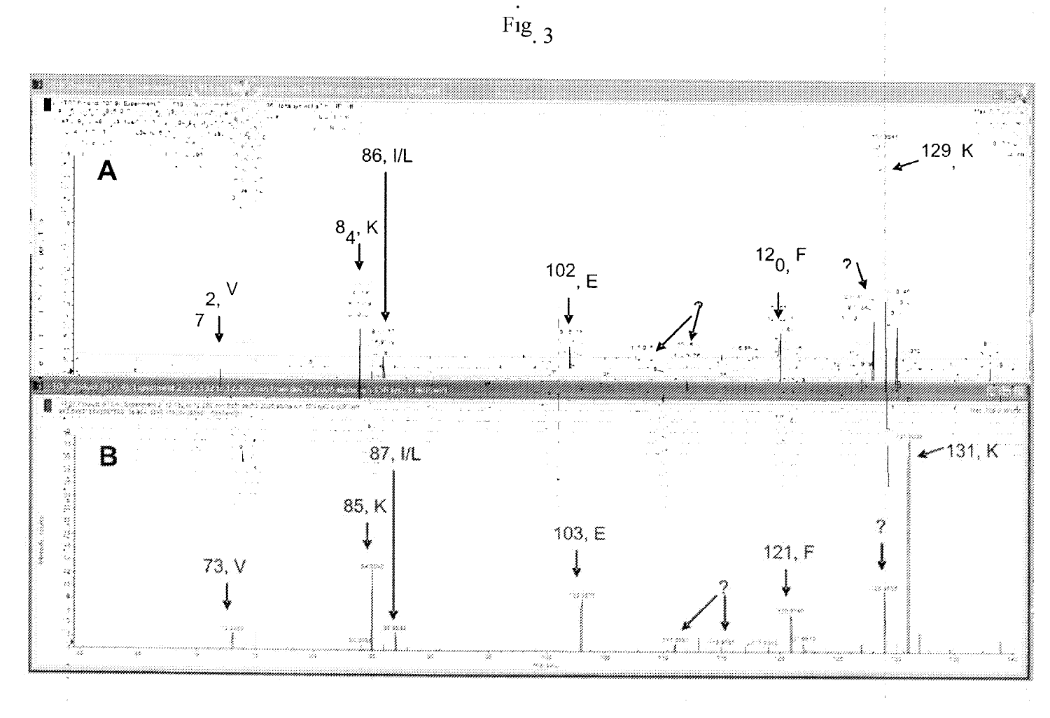

FIG. 2 is an MS scan of a sample of MHC-derived peptides isolated from a culture of antigen presenting cells. Within that group of peptides, a target epitope derived from an exogenous antigen cannot be identified using traditional techniques by mass alone because the sequence of the peptide epitope cannot be predicted and one single unique peak cannot be identified from the thousands of peptide sequences in the mixture. Also given the heterogeneity of MHC-bound peptides, predicting all potential epitopes from the antigen is virtually impossible when length heterogeneity (8-15 amino acids for MHC-I ligands and 9-33 amino acid for MHC II ligands) and post-translational modifications (>150 potential modifications) are considered.

In this example, an antigen is the molecule of interest and the detection of labeled MHC bound peptides derived from the antigen is performed by the method of the invention to discriminate such peptides of unknown size and composition from other MHC-derived peptides isolated from the culture. To perform the method of the invention, an isotopically labeled exogenous source of the antigen of interest is created as described herein and processed into peptides by the antigen presenting cells. The resulting naturally processed precursor peptides, while not recognizable in a conventional scan, are identified by the unique isotopic shift observed in a precursor ion scan specific for low molecular weight fragment product ions, followed by mass analysis of precursor mass that yielded the characteristic isotope-labeled product ions.

Referring to FIGS. 3A and 3B, in this example, an ¹⁵N labeled antigen is produced using conventional recombinant expression techniques. The resulting ¹⁵N-labeled precursor peptide yields unique immonium product ions characteristic of the ¹⁵N-labeled amino acids of which the peptide is composed. (See Table 1.) Although some data can be acquired ex post facto by analyzing an entire set of ms/ms data, in the practice the invention, the isotopic shift in the product ions is detected from the precursor ion scan in real time and used to selectively screen for the ¹⁵N peptides also preferably in real time. In practice, the mass of the selected isotope used to create the isotope labeled molecule of interest is known based on the isotope selected by the researcher practicing the method. Based on the isotope selected, the characteristic isotopic shift is known and the peak generated by a product ion is determined. In the example above, the selection of ¹⁵N yields the unique immonium as listed in Table 1 above. At least one low mass product immonium ion characteristic of the isotopic shift conveyed by the ¹⁵N peptide is monitored. All immonium ions contain at least one nitrogen and hence the mass of the ¹⁵N immonium ion will be shifted by at least one Dalton. This characteristic isotopic shift is illustrated in FIGS. 3A and 3B for labeled and unlabeled peptides.

The method of the invention may be practical by acquiring a precursor ion scan using a current generation triple quadrupole mass spectrometer, where a single fragment ion is targeted and then scan for precursor masses that give rise to the target ion. This is also shown in FIGS. 3A and 3B, where scanning of the peptide mixture using the mass of the ¹⁵N immonium ion of glutamate and glutamine at m/z 103. Peptides that contained ¹⁵N glutamate and/or glutamine are screened out of the mixture. When screening for naturally processed peptides there is no guarantee that the target peptide contains glutamate and/or glutamine and they may be missed in the scan.

Referring again to FIG. 3, a current generation triple quadrupole mass spectrometer targets product ion exhibiting the unique isotopic shift resulting from the selected isotope and then scans for precursor masses that give rise to the target ion. This is shown in FIGS. 3A and 3B, where scanning of the peptide mixture using the mass of the ¹⁵N immonium ion of glutamate and Glutamine at m/z 103. Precursor peptide masses that contained ¹⁵N glutamate and/or Glutamine are screened out of the mixture. However, when screening for naturally processed peptides there is no guarantee that the target peptide contains glutamate and/or glutamine and they may be missed in the scan.

Alternatively, if there are several target peptides only some (the glutamate containing peptides) will be observed. Thus, in the practice of the present invention, simultaneous monitoring of a plurality of product ions exhibiting the isotopic shift will provide a much greater chance of identifying all of the labeled peptides in the mixture. An additional benefit of this would be that by monitoring multiple target product ions, one reduces the possibility of false positives due to chemical noise, difference in peptide concentrations in the sample, or other contaminants in the sample.

A software algorithm permits in part of the selected isotope or otherwise recognized the characteristic isotopic shift caused by the labeling reaction. The software monitors the ratio of the regular ion peak intensity and the corresponding intensity of the ¹⁵N (or other isotope) precursor ion masses that are isotopically labeled. For each target product ion, the isotopic ratio would describe the difference between the intensity of the unlabeled mass and the first isotope peak which can be predicted from isotopic abundance. For a regular low mass ion from a molecule (e.g. peptide) the ratio would be very high e.g. 97:1. For a ¹⁵N peptide the ratio would dramatically change e.g. 1:90. Importantly, using this method it would also be possible to identify the presence of a labeled peptide even if one or more unlabeled co-eluting isobaric peptides was present in the mixture because the ratio would still be skewed e.g. 85:15.

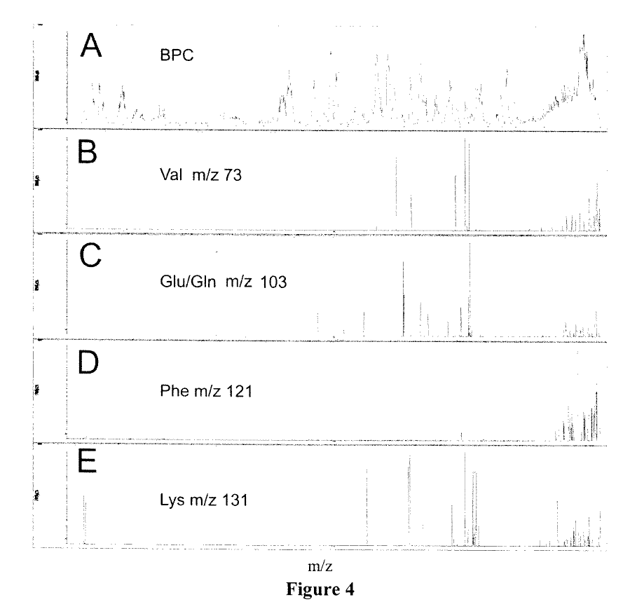

Referring to FIG. 4, using ¹⁵N labeled immonium ions as target product ions, peptides are selectively identified that are derived from a ¹⁵N labeled protein, in preference to peptides from a second unlabeled protein in the same mixture. As shown in FIG. 4, the mixture contained a large number of LysC peptides from both BSA and ¹⁵N labeled α-synuclein. However, a precursor ion scan detected the target product ions that had a characteristic ¹⁵N immonium ion and the BSA peptides were selectively excluded in favour of the α-synuclein peptides.

Thus, another advantage of the present invention is that the method effectively enables the user to filter out uninteresting ions and concentrate on molecules of interest.

More importantly, because the techniques do not artificially modify the molecule of interest (other than skewing the isotope abundance) the technique is ideally suited to the examination of the natural processing of proteins/peptides or other molecule in a biological context. In the case of proteins or peptides, unlike MRM or precursor ion experiments, the present invention makes no assumptions about the precursor or a product ion mass other than it contains amino acids. Thus, in a biological context where the protein or peptide can be processed or modified in unexpected ways, the resulting products can be can be identified by their distinctive isotope immonium ion masses.

A feature of this technique is the ability to examine the basic building blocks of the target molecule to sort the component ions. In the case of a peptide, because all peptide will contain amino acids, and hence will yield a set of immonium ions in the low mass region the particular set of product immonium ions will depend entirely on the constitution of the precursor peptide. A peptide that contains a valine will yield an immonium ion at m/z 72 (73 for ¹⁵N valine). A precursor scan could be used to monitor m/z 72 to identify valine containing peptides however peptides that do not contain valine will be ignored. By simultaneously observing the masses of all of the immonium ions both the valine and non-valine containing peptides will be detected. Thus, the detection of target peptides using the present method does not rely on prior knowledge of a specific precursor or product ion mass.

In a preferred embodiment, the present invention uses the low mass region of the product ion spectra to identify the target precursor masses. The low mass product ions are essentially represented by their unlabeled ions. The advantage of using the low mass region is that the low mass product ions are mainly represented by the regular (unlabeled) peaks with very little of the other isotope species present. By isotopically labeling a target product molecule to create the isotopic shift, the change in the isotopic ratio can be easily recognized. Thus, an isotopically labeled precursor mass can be identified by simply comparing the intensity (by a ratio or any other mathematical means) of the “normal” and “isotope” peaks for a given product ion or set thereof.

In certain circumstances, it is also possible to use the higher mass region of the product ion spectra to identify the target precursor mass ion(s). At the higher mass region of the spectra, the other isotope peaks become more abundant, but the mass difference between the ions and the presence of the isotopic label can still be observed in some cases.

Those skilled in the art will readily appreciate that the present invention can be used with other search tools, such as ProteinPilot™. One of the features of ProteinPilot is its ability to search for post translational modifications. Both MRM's and precursor scans, however, have underlying assumptions that exclude the identification of unexpected modified peptides. In contrast, the present invention makes no assumptions about the post translational state of the peptide, and can selectively screen out modified peptides that would otherwise be missed by an MRM.

In the practice of the method of the invention, it may be able to identify the shift in the ions (e.g. the shift in the pattern of immonium ions for a labeled peptide). Software analyzes the product ions for characteristic ions and then when a suitable precursor is detected the system switches scan type to further characterise the target.

### iv. Instrumentation

The methods of the present invention can be performed using tandem mass spectrometers and other mass spectrometers that have the ability to select and fragment molecular ions (such as an ion trap). Tandem mass spectrometers have the ability to select and fragment molecular ions according to their mass-to-charge (m/z) ratio, and then record the resulting fragment (product) ion spectra. More specifically, product fragment ion spectra can be generated by subjecting selected ions to dissociative energy levels (e.g. collision-induced dissociation (CID), ETD, ECD, IRMPD etc). For example, ions corresponding to labeled peptides of a particular m/z ratio can be selected from a first mass analysis, fragmented and re-analyzed in a second mass analysis. Representative instruments that can perform such tandem mass analysis include, but are not limited to, magnetic four-sector, tandem time-of-flight, triple quadrupole, ion-trap, hybrid quadrupole time-of-flight (Q-TOF), Fourier transform-ion cyclotron resonance, and orbitrap mass spectrometers.

These types of mass spectrometers may be used in conjunction with a variety of ionization sources, including, but not limited to, electrospray ionization (ESI) and matrix-assisted laser desorption ionization (MALDI). Ionization sources can be used to generate charged species for the first mass analysis where the analytes do not already possess a fixed charge. Additional mass spectrometry instruments and fragmentation methods include post-source decay in MALDI-MS instruments and high-energy CID using MALDI-TOF (time of flight)-TOF MS. For a recent review of tandem mass spectrometers please see: R. Aebersold and D. Goodlett, Mass Spectrometry in Proteomics. Chem. Rev. 101: 269-295 (2001).

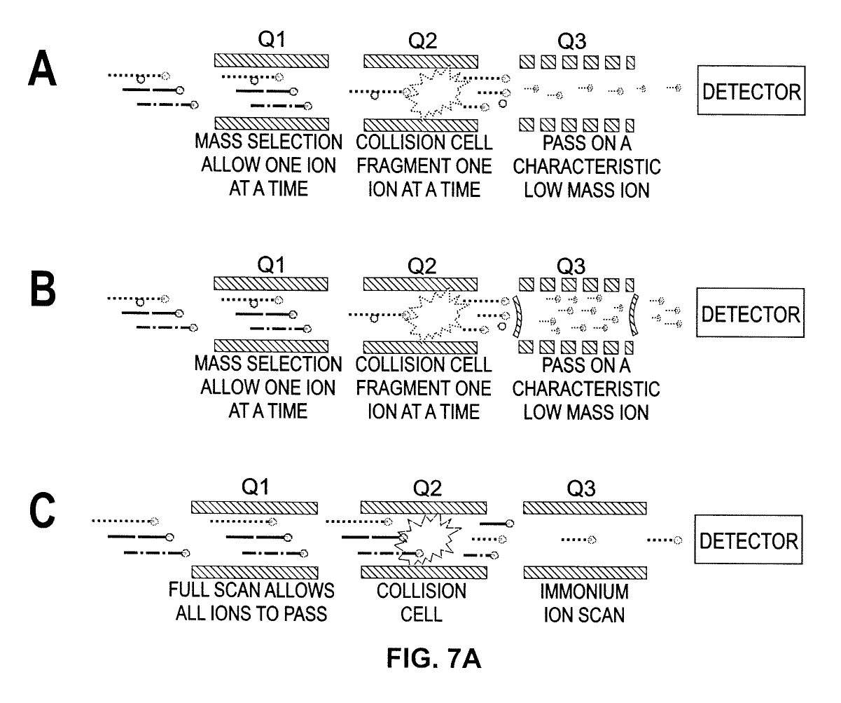

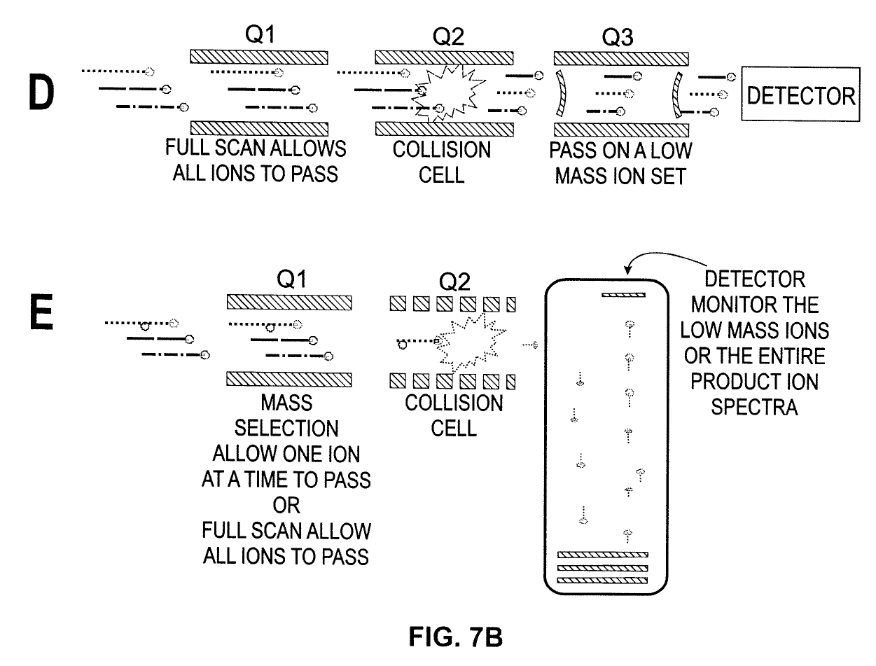

FIG. 7 is a schematic representation of possible alternative arrangements for performing methods according to the present invention. FIG. 7A represents a traditional precursor scan, however the target is an isotopically labeled low mass product ion that is distinct from other routinely (unlabeled) observerd product ions. FIG. 7B represents a variation on the idea of a precursor scan in a QTRAP instrument where the Q3 Trap stores ions corresponding to the low mass region and then scans them out to the detector.

### EXAMPLES

The following examples are intended to illustrate but not limit the present invention.

### Example 1

### Precursor Ion Scanning for ¹⁵N Immonium Ions of ¹⁵N α-Synuclein LysC Digest

A 1 mg per/ml BSA trypsin digest was spiked with and equal volume of 1 mg/ml ¹⁵N α-synuclein LysC digest. As shown in FIG. 4, A) the base peak chromatogram for the mixture indicating a complex mix of the two sets of peptides. The automated data acquisition software selected ions from both BSA and α-synuclein for MS/MS analysis. Using the same sample multiple precursor ion scan experiments were used to specifically target ¹⁵N labeled precursor peptide masses via immonium ions characteristic of a ¹⁵N labeled amino acid. The masses selected were m/z 73, 103, 121, and 131 corresponding to immonium ions for ¹⁵N Valine, ¹⁵N Glutamate/Glutamine, ¹⁵N phenylalanine, and ¹⁵N Lysine respectively. Referring again to FIG. 4, in each case B), C), D) and E) the scans selectively identified α-synuclein peptides as the molecule of interest and ignored BSA peptides (one false positive was recorded in the lysine 131 precursor scan). It is important to note each of the four immonium ion scans identified different sub-sets of the ¹⁵N α-synuclein peptides, demonstrating that a standard precursor ion scan would not identify all of the possible peptides in the mix. Hence, it is preferred to monitor as many ¹⁵N immonium ions as possible to ensure the best chance of identifying all of the target peptides. It should also be noted that a false positive, such as the single false positive in the ¹⁵N lysine 131 scan, can be avoided by using software to simultaneously monitor the presence of other ¹⁵N immonium ions in real time, thereby providing additional evidence of the presence of a target precursor peptide. Additionally, the software may also monitor the ratio of the regular versus the ¹⁵N immonium ion to reduce the possibility of false positives that arise from the natural isotope abundance in the unlabeled peptides as described above. In such a case, the intensity of a given immonium ion must be high enough to surpass a threshold value that distinguishes the natural isotopic abundance and does not trigger a regular precursor ion scan.

### Example 2

### Identification of the ¹⁵N Peptide EGVLYVGSK by Precursor Ion Scanning for ¹⁵N Immonium Ions

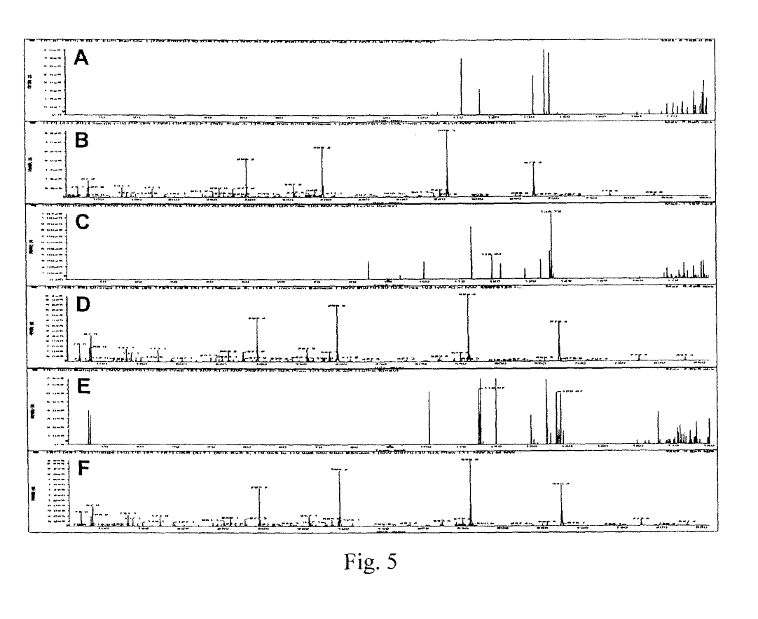

Referring to FIG. 5A-5F, 1 mg per/ml BSA trypsin digest was spiked with and equal volume of 1 mg/ml ¹⁵N α-synuclein LysC digest. As shown in FIG. 5, precursor scans (A, C, and E) for ¹⁵N Val (m/z 73), ¹⁵N Glu/Gln (m/z 103) and ¹⁵N Lys (m/z 131) all identified the same ¹⁵N peptide EGVLYVGSK (B, D, and F).

### Example 3

### Selective Detection of the ¹⁵N Peptide QGVAEAAGK

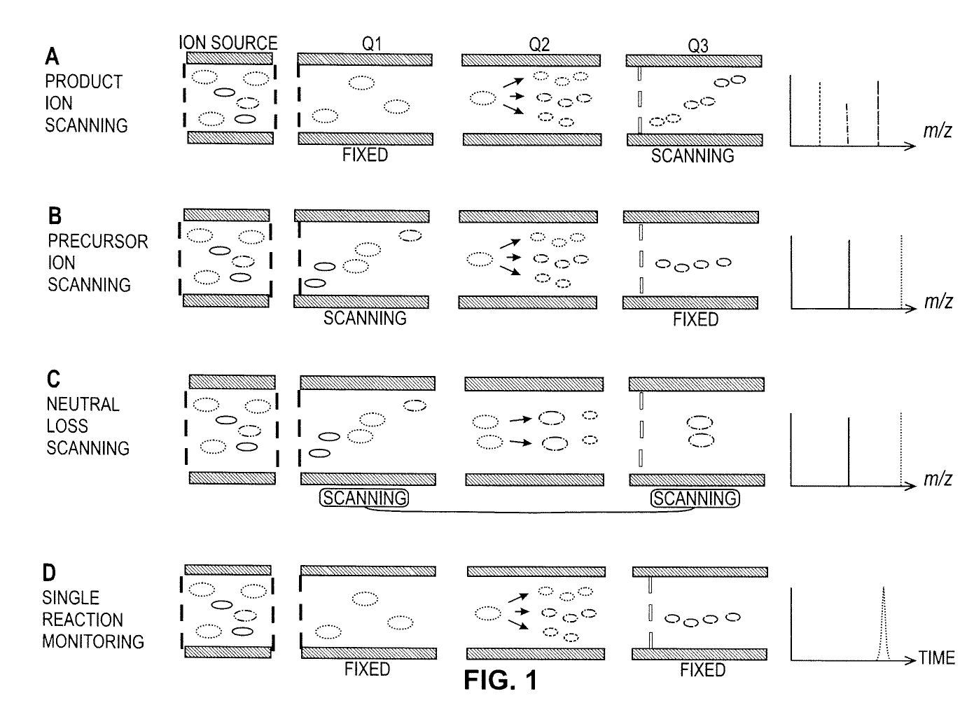

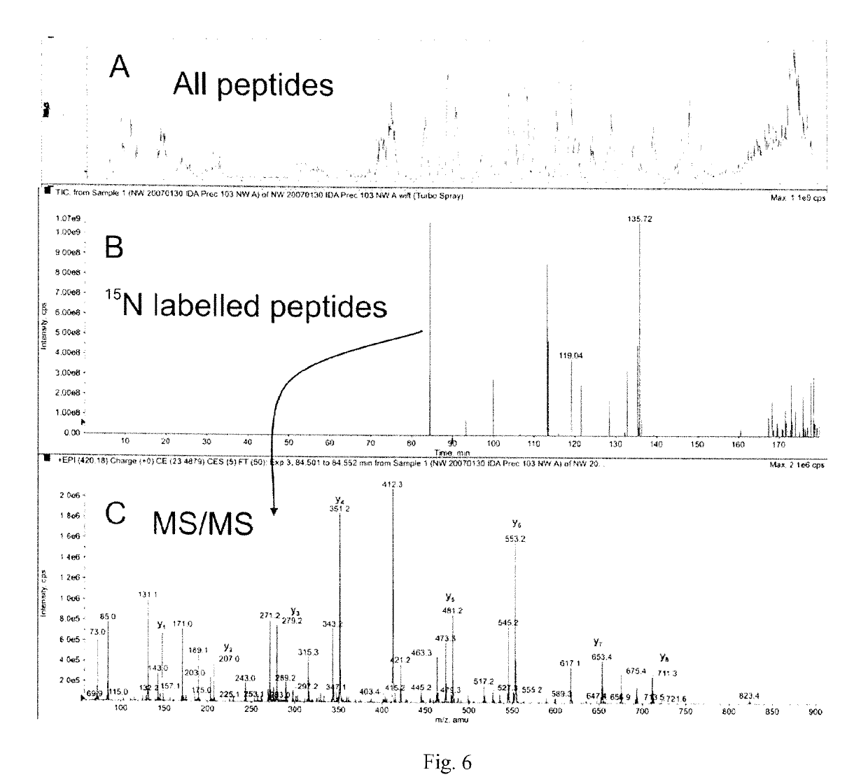

Referring to FIGS. 6A and 6B, 1 mg per/ml BSA trypsin digest was spiked with and equal volume of 1 mg/ml ¹⁵N α-synuclein LysC digest. As illustrated in FIG. 6 (A) The TIC for the precursor scan for m/z 103 and (FIG. 6B) the MS/MS result for the ¹⁵N labeled peptide QGVAEAAGK. The peptide QGVAEAAGK was only detected in the precursor scan for the product ion ¹⁵N Glu/Gln (m/z 103), despite the fact that the peptide also contained valine and lysine (and therefore should have been detected in the ¹⁵N Val and Lys scans). This demonstrates the benefit of using multiple target product ions to detect the peptides contained in the sample.

### Example 4

### Peptide Epitope Identification

Any isotope labeled molecule of interest, such as ¹⁵N-Labeled exogenous antigen, can be incubated with a suitable antigen presenting cell for 48 hours to allow uptake, processing and presentation of labeled peptide/s. The cells are washed (to remove excess antigen) and then pelleted. Cell pellets are then resuspended in 0.5% TFA which results in cell lysis and precipitation of most cellular proteins. The acid wash also releases MHC bound peptides which are small enough to remain soluble in the acid solution. The mixture is then centrifuged to pellet the protein and cell debris, leaving an acid solution containing the previously MHC bound peptide. Remaining high molecular weight contaminants are removed by ultrafiltration (e.g. a 5 kD MWCO filter). The resulting peptide fraction is then lyophilized to reduce the sample volume, and then loaded onto an LC/MS/MS system for peptide separation and analysis (e.g. using a 0.3 mm×5 mm C18 reversed phase HPLC trap column and a 0.075 mm×10 cm C18 resolving column, with an water/acetonitrile/0.1% Formic acid gradient). The eluting peptides can be monitored for ¹⁵N content in real time (by observing the low mass immonium product ions) using the precursor ion scan as detailed above as peptide precursors masses are identified, a regular product ion scan is triggered to identify the sequence of the precursor and hence the presented peptide epitope from the exogenous antigen. When a peptide precursor mass is identified and subjected to further mass analysis pursuant to the present method to obtain the amino acid sequence of the peptide, the present method includes adjusting the automated computational MS sequencing techniques to scan for the presence of isotope labeled amino acids in the peptide, for example by using the residue mass values for ¹⁵N labeled amino acids.

### Example 5

### Monitoring the Metabolites of a Protein Pharmaceutical

Created as an isotope-labeled molecule of interest where the in vivo metabolic processes constitute an assay of the present invention by virtue of the potential to modify the labeled protein as described herein. Serum samples can then be taken at specific time points. Each sample is acidified with 0.5% TFA or 80% acetonitrile, resulting in precipitation of most proteins. Most of the serum peptides are small enough to remain soluble in the acid or acetonitrile solution. The mixture is then centrifuged to pellet the protein, leaving a solution containing the serum peptides. Remaining high molecular weight contaminants can be removed by ultrafiltration (e.g. a 5 kD MWCO filter). The resulting peptide fraction is then lyophilized to reduce the sample volume, and then loaded onto an LC/MS/MS system for peptide separation and analysis (e.g. using a 0.3 mm×5 mm C18 reversed phase HPLC trap column and a 0.075 mm×10 cm C18 resolving column, with an water/acetonitrile/0.1% Formic acid gradient). The eluting precursor peptides can be monitored for ¹⁵N content (by observing the low mass immonium ions) in real time according to the method of the present invention. As peptide precursor masses are identified, a regular product ion scan can be triggered to identify the sequence of the precursor mass and hence peptides derived from the original pharmaceutical molecule of interest.

### Example 6

### Monitoring for Cell Derived Biomarkers

Tumor or other cells can be propagated in ¹⁵N cell culture media for several generations to ensure a high level of incorporation of the ¹⁵N label. In one example, ¹⁵N-labeled tumor cells can then be harvested and injected into the peritoneal cavity of a rat where they can form a tumour cell mass. Serum samples are then taken at specific time points. Alternatively tumor cells could be introduced intravenously and metastatic colonisation of the lung and other tissues examined and cell derived biomarkers screened either in the plasma or other fluids that drain the affected sites. In either event, metastasis or formation of the tumor cell mass modified the structure of components of the tumor cells and constitutes an assay of the present invention.

A variety of different experiments can be performed using the ability to monitor low mass product ions by the precursor ion scan. For example, the serum could be screened for ¹⁵N labeled peptides as per example 5. Also several other molecules can also be screened for, such as carbohydrates (where, for example, ¹⁸O or ¹³C labelling would be more effective), lipids (where, for example, ¹⁸O or ¹³C labelling more effective), DNA or RNA (where, for example, ¹⁸O or ³²P labelling could be used), and other metabolites.

The advantages of this strategy are (i) host and cell derived responses can be easily delineated by comparing results from mass spectrometric analysis according to methods of the present invention to traditional proteomics analyses of protein expression and (ii) a variety of cells (tumor cells, tissue specific cells (e.g. pancreatic beta-cells), can be introduced to any experimentally tractable organism (mice, rats, non-human primates etc).

### Example 7

### Screening for Unexpected or Unknown Post Translational Modifications

Screening for unexpected or unknown post translational modifications in a molecule of interest is achieved by adding an ¹⁵N-labeled substrate protein to a biological mixture. The mixture can contain an enzyme activity that post-translationally modifies the protein in an unknown way. The substitute protein is allowed to incubate for 1 hour. Trypsin can then be added to the mixture to cut all of the proteins into peptides. The peptides can then be then loaded onto an LC/MS/MS system for peptide separation and analysis (e.g. using a 0.3 mm×5 mm C18 reversed phase HPLC trap column and a 0.075 mm×10 cm C18 resolving column, with an water/acetonitrile/0.1% formic acid gradient). The eluting peptides can be monitored for ¹⁵N content (i.e. observing the low mass immonium ions) using the precursor ion scan according to the method of the present invention. As peptide precursor masses are identified, a regular product ion scan can be triggered to identify the sequence of the precursor and peptides derived from the original protein. In this case, any modified precursor peptides will be identified regardless of how they have been post translationally modified. This provides a distinct advantage over an MRM or standard precursor scanning experiment as no prior knowledge (or guess) is required (other than it will contain ¹⁵N amino acids and therefore ¹⁵N immonium ions) for the identification of the modified peptide and there is no need to attempt to estimate the nature of any modification that might have occurred, i.e. phosphorylation, methylation, or trimethylation, etc.

As noted above, the practice of the present invention is possible with any current generation mass analysis device. Most modern devices are controlled by software that controls the operational setting and parameters of the device and control the handling of samples and the various steps involved in analyzing a sample.

In practical use, the precursor ion scan detects a selected ion or group of ions that exhibit a characteristic isotopic shift. Accordingly, a user may use a mass instrument interface to input a particular ion or isotope to direct the specific product ions to be detected in the precursor ion scan. The isotope and/or selection may also direct the instrument to select specific product ions for detection. The instrument may maintain values, such as those in Tables 1 and 2 above in memory, i.e., as a set of look up values or otherwise, to facilitate the selection of an ion or isotope of specific values, or a portion of the mass spectrum, such as the low mass region described above for focused analysis. It is also accepted for the instrument to have a default ion or isotope selection, such as the ¹⁵N immonium ion.

Once the instrument begins to perform the precursor ion scan the software monitors and/or displays the intensity of the selected product ion peak to detect the characteristic isotopic shift. As described above, an intensity threshold may be used to distinguish product ion peaks in the precursor ion scan. If used, the threshold value can be used as a trigger for the software to direct the instrument to perform an enhanced resolution scan of the identified precursor mass or a product ion scan in either order. Depending on the configuration of the instrument used. The software may be designed to collect data on one or a plurality of precursor ion scans prior to the enhanced resolution scan. Similarly, all precursor ions so identified may be subjected to the enhanced resolution scan individually as they are identified by each precursor ion scan(s) or the identified precursor messes may simply be flagged for further analysis after completion of the entire set of precursor ion scans is complete.

Again, depending upon the specific design and user selected parameters of the instrumentation used, the precursor ion scan may monitor and or display the intensity of two or more characteristic product ions. Two or more characteristic product ions may be detected in the scan of one or more precursor messes and the resulting intensity data may be used to direct the further operation of the instrument in several ways. When the two product ions exhibiting the characteristic isotopic shift are detected in a single precursor mass, that precursor mass may be prioritized for the enhanced resolution scan and/or the regular product ion scan. Similarly, the use of a plurality of product ions in a precursor ion scan may be specified where, for example, two different isotopes are used to create the isotope labeled version of the molecule of interest.

The step of monitoring the characteristic isotopic shift in the precursor ion may be accompanied by the step of monitoring the abundance or intensity of the non-labeled or non-isotopic product ion to determine whether or not the precursor mass was actually labeled. Under circumstances where the unlabeled peptide is present in high abundance, a false positive can be a result. The measurement of both the isotope labeled and non-isotope labeled ions can be used to distinguish whether or not the precursor mass is derived from the isotope label molecule originally created. Where the system determines that the precursor mass does not contain the isotope from the isotope labeled molecule of interest, the analysis of the precursor mass is suspended and the system advances to the next peak in the precursor ion scan where the process is repeated.

In a QqTOF instrument such as the Applied Biosystems QSTAR Elite, the capability exists to monitor the intensity of all product ions, or a substantial number thereof, to determine if the intensity of one of more is above a given threshold. In such case, then each target precursor mass so identified is selected for the TOF Scan of the identified precursor, optionally followed by regular product ion scan of the identified precursor. As above, the regular non-isotope equivalent of the monitored ion can be used to compare between the two species to determine whether or not any detected ion resulted from the original isotopic labeling of the molecule of interest. The comparison of the isotope and non-isotope versions of the product ions is only one example of any of a variety of mathematical techniques known to those skilled in the art that can be used to distinguish the product ions from an isotopically labeled precursor by those produced by an un-labeled molecule.

In each instance, the software may readily be configured to analyze a single peak or a series of peaks generated by the precursor ion scan to apply a number of analytical parameters to each peak, with the ability to identify whether or not the intensity values for the peak, and/or there relative intensity compared to another peak, indicate that the precursor mass should be subjected to further analysis. The operational design of the software, again depending on the configuration of the instrument, may automatically instruct the system to proceed sequentially through a number of precursor ion scan peaks to exhaust all the available precursor masses available in the analyte sample. As in any modern mass analysis instrument, the analytical parameters of the instrument may be selected by the user in accord with the sample and/or the designs of the individual experiment. The following listing is a representative sample of instrument parameters used on the QSTAR instrument for an analysis substantially similar to that presented in the examples above.

File Information for Sample 1 (May 21, 2008 CR 15 min serum stab teab 20 ul neat pre85ce90) of May 21, 2008 CR 15 min serum stab teab 20 ul neat pre85ce90.wiff

| | |
|---|---|
| File Name: | May 21 2008 CR 15 min serum stab teab 20 ul neat |
| | pre85ce90.wiff |
| File Path: | G:\QSTAR data\may 21 charles proins\ |
| Original Name: | May 21 2008 CR 15 min serum stab teab 20 ul neat |
| | pre85ce90.wiff |
| Software Version: | Analyst QS 2.0 |

Log Information from Devices at Start of acquisition:

| | | |
|---|---|---|
| Software Application | Tempo LC device CH2 | 1 |
| Software Application | Tempo nano LC Autosampler | 2 |
| Software Application | Tempo LC device CH1 | 0 |
| Tempo LC device CH1 | User=QSTARELITE\Administrator | |
| | Computer=QSTARELITE | |
| Eksigent Software v2.06 | | |
| Build 051115 Firmware | 2.39 | |
| Tempo LC device CH2 | User=QSTARELITE\Administrator | |
| | Computer=QSTARELITE | |
| Eksigent Software v2.06 | | |
| Build 051115 Firmware | 2.39 | |
| Tempo LC device CH1 | User=QSTARELITE\Administrator | |
| | Computer=QSTARELITE | |
| Active Method | ch1 7 min 5ul min elite | |
| Duration | 7 min | |
| Ref. Flowrate | 5000 nL/min | |
| Constant Flow | True | |
| Preflush | True | |
| Postflush | False | |
| Tempo LC device CH2 | User=QSTARELITE\Administrator | |
| | Computer=QSTARELITE | |
| Active Method | ch2 5-50 b over 5-55 min 300 nl min 70 min total elite | |
| Duration | 70 min | |
| Ref. Flowrate | 300 nL/min | |
| Constant Flow | True | |
| Preflush | True | |
| Postflush | False | |
| Mass Spectrometer | QSTAR Elite System | 0 |
| Config Table Version | 01 | |
| Firmware Version | M401402 B4T0301 M3L1415 B3T0300 | |
| Component Name | Hybrid Quadrupole-TOF LC/MS/MS Mass Spectrometer | |
| Component ID | QSTAR Elite | |
| Manufacturer | AB Sciex Instruments | |
| Model | 1017064/Q | |
| Serial Number | AP11240706 | |
| Source Housing | Nanospray | |
| Tempo nano LC Autosampler | User=QSTARELITE\Administrator | |
| | Computer=QSTARELITE | |
| Eksigent AS1 v1.0 | | |
| Firmware | 100 Firmware | microEndurance protocol 1.4 |
| Mass Spectrometer | QSTAR Elite System | 0 |
| Start of Run —Detailed Status | | |
| Vacuum Status | At Pressure | |
| Vacuum Gauge (10e−5 Torr) | 4.3 | |
| Backing Pump | Ok | |
| Q1 Turbo Pump | Normal | |
| Q2/TOF Turbo Pump | Normal | |
| Sample Introduction Status Ready | | |
| Source/Ion Path Electronics | On | |
| Source Type | Nanospray | |
| Source Temperature | Not Applicable | |
| Source Exhaust Pump | Ok | |
| Injection Manifold | Inject | |
| Tempo nano LC Autosampler | User=QSTARELITE\Administrator | |
| | Computer=QSTARELITE | |
| Active Method | 20ul sample_trap_20 min gradient_off elite | |
| Sample Name | May 21 2008 CR 15 min serum stab teab 20 ul neat pre85ce90 | |
| Sample ID | | |
| Sample Row | D | |
| Sample Column | 2 Time from start = 0.0167 min | |
| Mass Spectrometer | QSTAR Elite System | 0 |
| End of Run —Detailed Status | | |
| Vacuum Status | At Pressure | |
| Vacuum Gauge (10e−5 Torr) | 4.2 | |
| Backing Pump | Ok | |
| Q1 Turbo Pump | Normal | |
| Q2/TOF Turbo Pump | Normal | |
| Sample Introduction Status Ready | | |
| Source/Ion Path Electronics | On | |
| Source Type | Nanospray | |
| Source Temperature | Not Applicable | |
| Source Exhaust Pump | Ok | |
| Injection Manifold | Inject | |
| Time from start = 83.9000 min | | |

### Acquisition Info

Pulser frequency has been adjusted to the value of 6.991 kHz for this method.
Pulse 1 Duration was 14 μs for this method.
File has been acquired with TDCx8.

| Acquisition Method: | \NIIPe scan 85 plus tof scan unit res |
|---|---|
| | 70 MIN.dam |
| Acquisition Path: | D:\Analyst Data\Projects\API |
| | Instrument\Acquisition Methods\ |
| First Sample Started: | Thursday, 22 May 2008 8:10:00 PM |
| Last Sample Finished: | Thursday, 22 May 2008 8:10:00 PM |
| Sample Acq Time: | Thursday, 22 May 2008 8:10:00 PM |
| Sample Acq Duration: | 75 min 12 sec |
| Number of Scans: | 0 |
| Periods in File: | 1 |
| Batch Name: | \New Batch.dab |
| Batch Path: | D:\Analyst Data\Projects\API |
| | Instrument\Batch\ |
| Submitted by: | QSTARELITE\Administrator( ) |
| Logged-on User: | QSTARELITE\Administrator |
| Synchronization Mode: | LC Sync |
| Auto-Equilibration: | Off |
| Comment: | |
| Software Version: | Analyst QS 2.0 |
| Set Name: | May 21 2008 CR 15 min serum stab teab |
| | 20 ul neat pre85ce90 |
| Sample Name | May 21 2008 CR 15 min serum stab teab |
| | 20 ul neat pre85ce90 |
| Sample ID | |
| Sample Comments: | |
| Autosampler Vial: | 26 |
| Rack Code: | 48 vial |
| Rack Position: | 1 |
| Plate Code: | 48 vial |
| Plate Position | 1 |

### Software Application Properties

| | |
|---|---|
| Display Name: | Tempo LC device CH1 |
| Identifier Key: | {F22028A9-4FAB-49DC-BF68-E975F73AA1F2} |
| Method Filename: | C:\Program Files\Eksigent |
| | NanoLC\settings\method\CH1 7 min |
| | 5uL min elite.ini |

### Software Application Properties

| | |
|---|---|
| Display Name: | Tempo LC device CH2 |
| Identifier Key: | {F22028A9-4FAB-49DC-BF68-E975F73AA1F3} |
| Method Filename: | C:\Program Files\Eksigent |
| | NanoLC\settings\method\CH2 5-50 B |
| | over 5-55 min 300 nl min 70 min total elite.ini |

### Software Application Properties

| | |
|---|---|
| Display Name: | Tempo nano LC Autosampler |
| Identifier Key: | {977F44CE-40B7-4BDA-9CF7-A0D87CDE108A} |
| Method Filename: | C:\Program Files\Eksigent |
| | NanoLC\settings\EKAS1\20uL |
| | sample_trap_20 min gradient_off elite.ini |

### Quantitation Information:

| | |
|---|---|
| Sample Type: | Unknown |
| Dilution Factor: | 1.000000 |
| Custom Data: | |

### Quantitation Table:

Period: 1

| Duration: | 75.204 | mins |
|---|---|---|
| Cycle Time: | 14.7942 | secs |
| # Cycles: | 305 | |
| Period Delay: | 0.00 | secs |

Period: 1 Experiment: 1

| Scan Mode: | Profile | |
|---|---|---|
| Scan Type: | Positive Precursor Ion | |
| Resolution Q1: | UNIT | |
| Intensity Thres.: | 0 | counts |
| Settling Time: | 0.000 | ms |
| MR Pause: | 0.572 | ms |

| | |
|---|---|
| MCA: | No |
| GS1: | 25.00 |
| GS2: | 0.00 |
| CUR: | 25.00 |
| IS: | 2700.00 |
| IHT: | 90.00 |
| TOF Masses (Da): | Min = 59.0000 Max = 135.0000 |
| Accumulation Time (sec): | 0.0250 |
| Time Bins to Sum: | 10 Store full product mass range. |
| Channels: | 1 2 3 4 |

Precursors of:

| Center (Da) | Width (Da) | Enhance |
|---|---|---|
| 85.0000 | 0.5000 | Yes |
| Step Size: | 1.00 Da | |

| Start (Da) | Stop (Da) | Time (sec) | Param | Start | Stop |
|---|---|---|---|---|---|
| 400.00 | 950.00 | 13.7937 | DP | 90.00 | 90.00 |
| | | | FP | 90.00 | 90.00 |
| | | | DP2 | 40.00 | 40.00 |
| | | | CE | 90.00 | 90.00 |
| | | | CAD | 5.00 | 5.00 |
| | | | IRD | 28.58 | 28.58 |
| | | | IRW | 12.45 | 12.45 |

Period: 1 Experiment: 2

| Scan Mode: | None | |
|---|---|---|
| Scan Type: | Positive TOF MS | |
| Intensity Thres.: | 1 | counts |
| Settling Time: | 0.000 | ms |
| MR Pause: | 0.572 | ms |

| MCA: | No |
|---|---|
| GS1: | 25.00 |
| GS2: | 0.00 |
| CUR: | 25.00 |
| IS: | 2700.00 |
| IHT: | 90.00 |
| TOF Masses (Da): | Min = 400.0000 Max = 2000.0000 |
| Accumulation Time (sec): | 1.0001 |
| Time Bins to Sum: | 4 |
| Channels: | 1 2 3 4 Enhance All |

| Q1 Mass (Da) | % Time | Param | Start | Stop |
|---|---|---|---|---|
| 380.00 | 33.3 | DP | 90.00 | 90.00 |
| | | FP | 90.00 | 90.00 |
| | | DP2 | 40.00 | 40.00 |
| | | CAD | 5.00 | 5.00 |
| | | IRD | 75.93 | 75.93 |
| | | IRW | 33.07 | 33.07 |
| 600.00 | 33.3 | DP | 90.00 | 90.00 |
| | | FP | 90.00 | 90.00 |
| | | DP2 | 40.00 | 40.00 |
| | | CAD | 5.00 | 5.00 |
| | | IRD | 107.39 | 107.39 |
| | | IRW | 46.77 | 46.77 |
| 1200.00 | 33.3 | DP | 90.00 | 90.00 |
| | | FP | 90.00 | 90.00 |
| | | DP2 | 40.00 | 40.00 |
| | | CAD | 5.00 | 5.00 |
| | | IRD | 124.93 | 124.93 |
| | | IRW | 66.14 | 66.14 |

### Resolution Tables

| | | |
|---|---|---|
| Quad 1 | Positive | Unit |

Last Modification Date Time: March 31, 2008 11:18:16

| | |
|---|---|
| IE1 | 1.000 |

| Mass (Da) | Offset Value |
|---|---|
| 58.000 | 0.023 |
| 170.000 | 0.010 |
| 900.000 | −0.103 |
| 2000.000 | −0.342 |

### Calibration Tables

| | | |
|---|---|---|
| Quad 1 | Positive | Unit Resolution |

Last Modification Date Time: March 20, 2008 10:13:55

| Mass (Da) | Dac Value |
|---|---|
| 59.050 | 560 |
| 175.133 | 1694 |
| 906.673 | 8848 |
| 2010.469 | 19643 |

### Instrument Parameters:

TOF Mass Calibration Parameters:

| Polarity | Slope | Delay (nsec) |
|---|---|---|
| Positive: | 3.595355308243927100e−004 | |
| | 4.993532234759165100e+000 | |

### Keyed Text:

File was created with the software version: Analyst QS 2.0

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of performing a mass analysis comprising:
(a) creating an isotope-labeled molecule by replacing a constituent atom of a molecule of interest with an isotope of the atom, wherein each difference in mass between the constituent atom and the isotope comprises an isotopic shift;
(b) reacting the isotope labeled molecule in a sample under assay conditions that modify the structure of the isotope labeled molecule;
(c) ionizing molecules in the sample mixture to yield product ions;
(d) performing a precursor ion scan to detect characteristic product ions exhibiting the isotopic shift and identifying a precursor mass containing the characteristic product ions; and
(e) performing a mass spectrometric analysis on the precursor mass.

2. The method of claim 1 further comprising analyzing the precursor ion scan to determine an intensity of the ions exhibiting the isotopic shift, wherein the step of analyzing the intensity comprises comparing the quantity of ions exhibiting an isotopic shift with a threshold intensity of the isotope.

3. (canceled)

4. The method of claim 3 wherein the threshold intensity is approximately the natural abundance of the isotope.

5. The method of claim 1 wherein the creating step comprises expressing a polypeptide by growing an expression host in a growth media enriched with the isotope to express an isotope-labeled polypeptide.

6. The method of claim 1 wherein the step of performing a precursor ion scan comprises detecting a plurality of characteristic product ions each exhibiting the isotopic shift.

7. The method of claim 1 wherein the product ions are immonium ions comprising ¹⁵N or ¹⁴C.

8. The method of claim 1 wherein the precursor ion scan encompasses a low mass region for fragments having a mass-to-charge ratio of less than about 250.

9. The method of claim 7 further comprising the step of correlating at least two characteristic product ions to a precursor mass.

10. The method of claim 7 further comprising prioritizing a first precursor mass for mass spectrometric analysis based on the detection of at least two characteristic product ions.

11. The method of claim 5 wherein the isotope-labeled polypeptide is reacted under the assay conditions that yield fragments of the isotope-labeled polypeptide in the sample.

12. The method of claim 5 wherein the precursor ion scan detects a characteristic ¹³N immonium ion fragment of the isotope-labeled peptide to identify a precursor mass comprising a portion of the isotope-labeled molecule.

13. The method of claim 5 wherein the precursor ion scan detects a characteristic ¹⁵N immonium related ion fragment of the isotope-labeled peptide to identify a precursor mass comprising a portion of the isotope-labeled molecule.

14. The method of claim 5 wherein the characteristic product ion is selected from the group consisting of the x1 ion, y1 ion, z1 ion, a1 ion, b1 ion and c1 ion or combinations thereof.

15.-25. (canceled)

26. A method of performing a mass analysis comprising:
(a) expressing an isotope-labeled polypeptide exhibiting an isotopic shift;
(b) reacting the polypeptide in an assay to modify structure of the isotope-labeled the polypeptide, wherein the modified polypeptide is contained in a sample mixture;
(c) ionizing the modified polypeptide in the sample mixture;
(d) performing a precursor ion scan to identify characteristic product ions exhibiting the isotopic shift; and
(e) identifying precursor polypeptide masses containing the characteristic product ions.

27. The method of claim 26 wherein the precursor ion scan encompasses a low mass region for fragments having a mass-to-charge ratio of less than about 250.

28. The method of claim 26 wherein the isotope-labeled polypeptide is selected from the group consisting of a protein, an antibody, a protein antigen, or combinations thereof.

29. The method of claim 26 further comprising analyzing the precursor ion scan to identify characteristic product ions comprised of isotope-labeled amino acids.

30. The method of claim 29 further comprising the intensity of the product ions to a threshold, wherein the step of analyzing the intensity comprises comparing the quantity of ions exhibiting an isotopic shift with a threshold intensity of the isotope.

31. The method of claim 30 wherein the threshold approximates the natural abundance of the isotope contained in the isotope-labeled amino acids.

32. The method of claim 26 wherein the isotope-labeled polypeptide is reacted under the assay conditions that yield fragments of the isotope-labeled polypeptide in the sample.

33. The method of claim 26 wherein the precursor ion scan detects a characteristic ¹⁵N immonium ion fragment of the isotope-labeled peptide to identify a precursor mass comprising a portion of the isotope-labeled molecule.

34. The method of claim 26 wherein the precursor ion scan detects a characteristic ¹⁵N immonium related ion fragment of the isotope-labeled peptide to identify a precursor mass comprising a portion of the isotope-labeled molecule.

35. The method of claim 26 wherein the characteristic product ion is selected from the group consisting of the x1 ion, y1 ion, z1 ion, a1 ion, b1 ion and c1 ion or combinations thereof.

36.-45. (canceled)

46. A computer-readable storage medium coupled to a triple quadrupole mass spectrometer for performing a mass analysis comprising:
(a) means for ionizing polypeptide fragments containing an isotope-labeled amino acid in a sample mixture, wherein the polypeptide has been created by replacing a constituent atom of an amino acid of a polypeptide of interest with an isotope of the atom, wherein each difference in mass between the constituent atom and the isotope comprises an isotopic shift; and
(b) means for performing a precursor ion scan to identify characteristic product ions containing the isotope-labeled amino acids; and
(c) means for analyzing the precursor ion scan to detect precursor polypeptide masses containing the characteristic product ion.

47. The computer-readable medium of claim 46 further comprising a means for switching the mode of operation of the mass spectrometer.
